(19) United States
(12) Patent Application Publication
(10) Pub. No.: US 2021/0308293 A1
(43) Pub. Date: Oct. 7, 2021

(54) SAFEGUARDED GERMICAL LIGHTING DEVICE

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 16/840,731

(22) Filed: Apr. 6, 2020

(51) Int. Cl. A61L 2/10; A61L 2/26

(52) U.S. Cl. CPC A61L 2/10; A61L 2/26; A61L 2202/11; A61L 2202/25; A61L 2209/12

(57) ABSTRACT

A lighting device comprises a light head, two light sources, and a safeguard mechanism. The light head houses the two light sources. The first light source emits predominantly ultraviolet (UV) light (<400 nm) for germicidal illumination, and the second light source emits predominantly visible light (>400 nm) for general lighting. The lighting device may operate in two modes. The first operation mode is the general lighting mode, where the second light source is on for providing general lighting. The second operation mode is the germicidal lighting mode, where the first light source is on for providing germicidal lighting. The safeguard mechanism prevents the first light source in the germicidal lighting mode from turning on and causing accidental human exposure to the UV light from the first light source.

### BACKGROUND

### Technical Field

The present disclosure pertains to the field of lighting devices and, more specifically, proposes a safeguarded germicidal lighting device.

### Description of Related Art

UV light is known to be effective in killing bacteria and viruses and thus has been used in germicidal lighting applications. It is also known that direct exposure of human skin to UV light may lead to skin cancer or other health-related issues. As a result, UV-based germicidal lighting device is generally placed in a concealed environment or at an upper room location to avoid directly shining on people. However, there are situations where it is necessary or desirable to use UV light to clean viruses and bacteria on a desktop surface at close distances. In these situations, the UV light should be turned on only when no one is around. This approach requires user compliance and is used in medical environments where users are properly trained in safety practices. In a residential setting with children, however, it is not practical to rely on user compliance with the safety practices of UV germicidal lighting. Hence a better approach, with a guaranteed safeguard against the misuse of UV germicidal lighting, is necessary.

The present disclosure introduces a dual-mode lighting device that can be used for general lighting as well as germicidal lighting. In the germicidal lighting mode, an automatic safeguard mechanism is provided to prevent direct human exposure to UV light.

### SUMMARY

In one aspect, the lighting device comprises a light head, two light sources, and a safeguard mechanism. The light head houses the two light sources. The first light source emits predominantly UV light (<400 nm) for germicidal illumination, and the second light source emits predominantly visible light (>400 nm) for general lighting. The lighting device may operate in two modes. The first operation mode is the general lighting mode. In this mode, only the second light source is on for providing general lighting. The second operation mode is the germicidal lighting mode. In this mode, the first light source is on for providing germicidal lighting. The safeguard mechanism is responsible for preventing the first light source in the germicidal lighting mode from turning on and causing accidental human exposure to the UV light from the first light source.

The two operation modes may be implemented by one switch with a two-stage setting, where Stage 1 is for the general lighting mode and Stage 2 is for the germicidal lighting mode. Alternatively, two switches may be used, where Switch 1 is used for turning on the general lighting mode and Switch 2 is used for turning on the germicidal lighting mode. Under such implementation, it is foreseeable that the first light source and the second light source may be turned on at the same time. This may be desirable for some applications. For example, for a hallway or stairwell fixture, it is necessary to have the second light source (visible light) on when the first light source (UV light) is on in order to provide sufficient lighting for a safety camera to function properly.

There are different implementations of the safeguard mechanism for turning on the first light source. In some embodiments, the safeguard mechanism may be a pressable switch such that the first light source turns on while a user presses the switch continuously. Once the user releases the switch, the first light source shuts off. With the safeguard mechanism thus implemented through a pressable switch, a user must consciously activate the first light source continuously, thus effectively preventing accidental use of the first light source and avoiding accidental human exposure to the UV light from the first light source.

In some other embodiments, the safeguard mechanism may be a motion sensor. The motion sensor automatically shuts off the first light source in the germicidal lighting mode upon detecting motion in the surrounding area of the lighting device. With the motion sensor as a safeguard mechanism, it shuts off the first light source when an individual comes too close to the light device in the germicidal mode, thus freeing parents from concerns of accidental exposure of their children to UV light. There is no restriction on the type of motion sensor. It may be IR based or microwave based or even sound based, so long as it provides adequate motion detection.

In some other embodiments, the safeguard mechanism may be a timer. The timer sets the on-time duration of the first light source in the germicidal lighting mode, e.g., to 15 minutes, thus protecting a user from being exposed to UV light for more than 15 minutes. Different safeguard mechanisms may be combined for a more effective safeguard. For example, a motion sensor can be used together with a timer for the present disclosure such that if a user comes near the light device when the first light source is on, the motion sensor would shut off the first light source. And the timer would shut off the first light source after the on-time duration expires even if no motion is detected during that time. There are two benefits of this combined safeguard approach. Firstly, it saves energy that would be lost due to unnecessary UV lighting. Secondly, it serves as a double safeguard in case the motion detector malfunctions.

When using narrow-band UV light as the first light source, no visible light is emitted from the first light source while it is on, since UV light (<400 nm) is outside the visible wavelength spectrum (>400 nm). This means that a user may not be able to tell whether the first light source is on by simply looking at the first light source. It is necessary to have another safeguard mechanism for informing the user whether the first light source is on or not. Therefore, in some embodiments, an LED indicator light may be used to indicate the on/off status of the first light source.

There are different options for the technology of the first and the second light sources. In some embodiments, the first light source may be UV LEDs, and the second light source may be regular white light LEDs. Moreover, the technology of the UV LEDs enables the creation of narrow band UV LEDs for a particular UV wavelength range. Thus, the first light source may further comprise a third light source and a fourth light source. While the third and the fourth light sources both emit predominantly UV light (<400 nm), the main wavelength of the third light source is higher than the main wavelength of the fourth light source. Furthermore, in some embodiments, the third light source may be UVA LEDs emitting predominantly light in 315-400 nm wavelength range and the fourth light source may be UVC LEDs emitting predominantly light in 100-280 nm wavelength range. It is known that UVA light and UVC light each have a killing effect on certain bacteria and viruses. By using both UVA LEDs for the third light source and UVC LEDs for the fourth light source, the germicidal effect of the light device of the present disclosure is maximized.

The germicidal effect of the UV light is proportional to the coverage area of the germicidal light source. Thus, the wider the UV light coverage area, the wider the area that is cleaned by the UV light. Therefore, in some embodiments, the light head may be of elongated shape and the first and the second light sources may be of elongated shape, in order to provide a wider germicidal area.

In some embodiments, the light device also has an upholder. One end of the upholder connects to the light head, and the upholder supports the weight of the light head. In some cases, the upholder will be mounted on a base such that the lighting device of the present disclosure may be a free-standing lighting fixture. In some other cases, the upholder may be a ceiling-mounted chain or pole such that the light device of the present disclosure may be a pendant-mounted lighting fixture.

The germicidal effect of the UV light is proportional to the intensity of the UV light source. When the energy consumption of the UV light source is kept constant, the intensity of the UV light source remains constant for any given distance. However, if the distance of the UV light source to the surface to be cleaned is decreased, the germicidal cleaning effect may be increased without increasing the energy consumption of the UV light source. Therefore, in some embodiments, the upholder may have an adjustable mechanical structure for adjusting the position of the light head, and in the germicidal lighting mode, the first light head may be positioned within 30 cm of the surface illuminated by the lighting device for more effective germicidal cleaning.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts an embodiment of the present disclosure in the form of an LED flashlight.

FIG. 2 schematically depicts an embodiment of the present disclosure in the form of a linear overbed lighting fixture.

FIG. 3 schematically depicts an embodiment of the present disclosure in the form of an LED desktop light.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting apparatuses having different form factors.

The present disclosure introduces a lighting device that comprises a light head, two light sources, and a motion sensor. The light head houses the two light sources. The first light source emits predominantly UV light (<400 nm) for germicidal illumination, and the second light source emits predominantly visible light (>400 nm) for general lighting. The lighting device may operate in two modes. The first operation mode is the general lighting mode, where the second light source is on for providing general lighting. The second operation mode is the germicidal lighting mode, where the first light source is on for providing germicidal lighting. The safeguard mechanism prevents the first light source in the germicidal lighting mode from turning on and causing accidental human exposure to the UV light from the first light source.

### Example Implementations

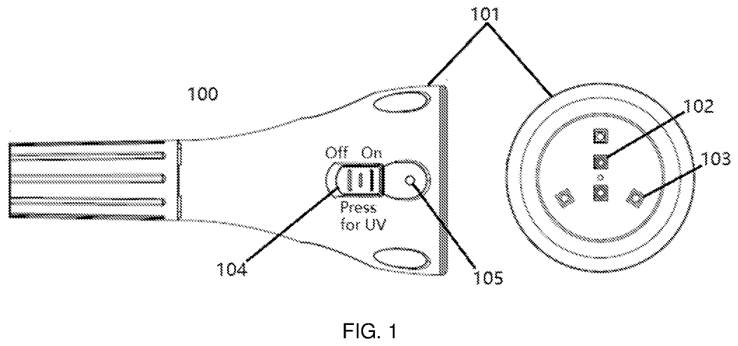

FIG. 1 shows an embodiment of the lighting device of the present disclosure in the form of an LED (Light Emitting Diode) flashlight 100. The light head 101 houses two light sources. The first light source 102 comprises two UV LEDs, and the second light source 103 comprises three white light LEDs. A user flips the switch 104 to turn on the second light source 103 for the general lighting mode operation. To activate the first light source 102 for the germicidal lighting mode operation, the user must keep pressing the switch 104. Once the user stops pressing the switch 104, the first light source 102 is turned off. Both the first light source 102 and the second light source 103 are on at the same time when the user is pressing the switch 104. When the switch 104 is at the OFF position, pressing the switch will not activate the first light source 102. The LED indicator 105 will light up when the first light source 102 is on, indicating to the user that the germicidal lighting is active. This embodiment can be used as regular flashlight, or as a germicidal lamp for cleaning a cellphone, a keychain, or any other smaller household items.

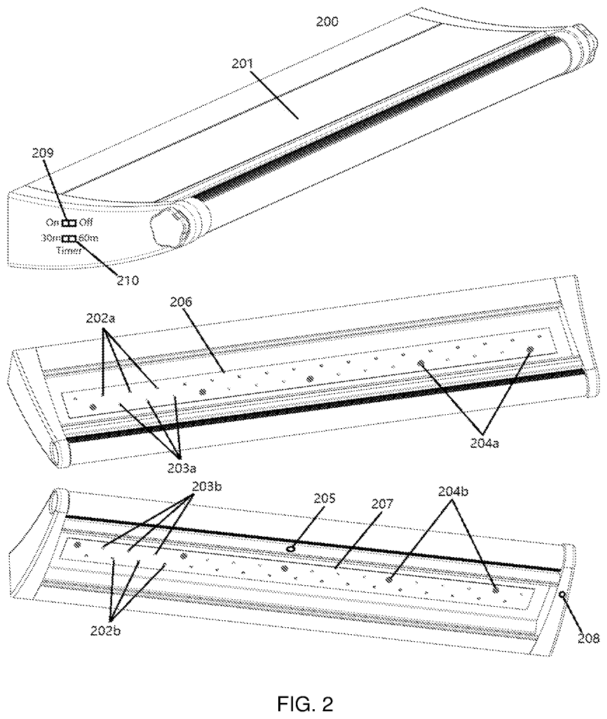

FIG. 2 shows an embodiment of the lighting device of the present disclosure in the form of an LED overbed light fixture 200 to be used in a patient room. The light head 201 has an up-light 206 and a down-light 207, and they each use three types of LEDs. The white light LEDs, 202a and 202b, produce visible light in the general lighting mode. The UVA LEDs 203a, 203b, and the UVC LEDs 204a, 204b, produce light in the germicidal lighting mode. The switch 209 can turn on either the white light LEDs (202a, 202b) for the general lighting mode or the UV LEDs (203a, 203b, 204a, 204b) for the germicidal lighting mode. The motion sensor 205 will shut off the UV LEDs upon detecting motion in the germicidal lighting mode, and the indicator LED light 208 informs the user whether the UV LEDs are on or not. The overbed light fixture has an elongated light head. The first light source, comprising 203a, 203b, 204a, 204b, and the second light source, comprising 202a, 202b, are also of elongated light shape, thus covering a wider area with germicidal lighting. This germicidal overbed light can provide effective bacteria and virus cleaning for a patient room when no one is in the room. Lastly, a user can use the timer 210 to set the duration of the germicidal lighting operation to be either 30 minutes for quick germicidal cleaning or 60 minutes for long germicidal cleaning.

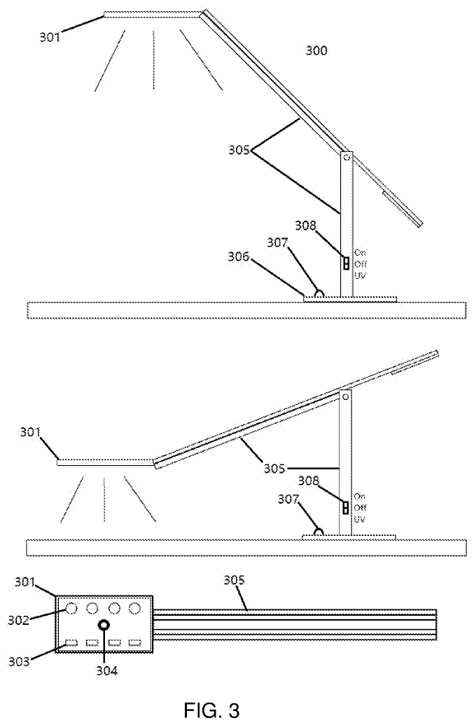

FIG. 3 shows an embodiment of the lighting device of the present disclosure in the form of an LED desktop light 300. The light head 301 has two light sources. The first light source 302 comprises UV LEDs, and the second light source comprises white light LED 303. The indicator light 307 is on when the first light source 302 is on, and is off when the first light source is off. The desktop light 300 has a configurable upholder 305, mounted on a base 306. One end of the configurable upholder 305 connects to the light head 301, and it supports the weight of the light head. In the germicidal mode, the first light source 302 is on, and the light head 301 can be positioned closer to the desktop surface by adjusting the configurable upholder 305 for effective killing of bacteria and viruses on the desktop surface. The switch 308 can be set to ON position for the general lighting mode or to UV position for the germicidal lighting mode. In the germicidal lighting mode, the motion sensor 304 will shut off the first light source 302 upon detecting motion. This germicidal desktop lamp can be used on any desktop or flat surface, and contribute to wellness into daily living whether at home or in the workplace.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A lighting device comprises:
a light head;
two light sources comprising a first light source and a second light source;
a safeguard mechanism;
wherein, in operation:
the light head houses two light sources,
the first light source emits an ultraviolet (UV) light (<400 nm) as a germicidal illumination,
the second light source emits a visible light (>400 nm) as a general lighting,
wherein, the lighting device has a first operation mode and a second operation mode, such that:
the first operation mode is a general lighting mode in which the second light source is on,
the second operation mode is a germicidal lighting mode in which the first light source is on, and
the safeguard mechanism prevents the first light source in the germicidal lighting mode from turning on to avoid causing accidental human exposure to the UV light from the first light source.

2. A lighting device of claim 1, wherein the safeguard mechanism comprises a pressable switch, and wherein the first light source turns on responsive to the switch being pressed continuously.

3. A lighting device of claim 1, wherein the safeguard mechanism comprises a motion sensor, and wherein the motion sensor shuts off the first light source in the germicidal lighting mode upon motion detection in a surrounding area of the lighting device.

4. A lighting device of claim 1, wherein the safeguard mechanism comprises a timer, and wherein the timer sets an on-time duration of the first light source in the germicidal lighting mode.

5. A lighting device of claim 1, further comprising an indicator light, wherein the indicator light indicates an on/off status of the first light source.

6. A lighting device of claim 1, wherein the first light source comprises UV light emitting diodes (LEDs), and wherein the second light source comprises white light LEDs.

7. A lighting device of claim 1, wherein the first light source further comprises a third light source and a fourth light source, wherein each of the third and fourth light sources emits a UV light (<400 nm), and wherein a main wavelength of the third light source is higher than a main wavelength of the fourth light source.

8. A lighting device of claim 7, wherein the third light source comprises ultraviolet A (UVA) light emitting diodes (LEDs) that emit a light in a wavelength range of 315 nm-400 nm, and wherein the fourth light source comprises UVC LEDs that emit a light in a wavelength range of 100 nm-280 nm.

9. A lighting device of claim 1, wherein the light head is of an elongated shape, and wherein the first and the second light sources are of an elongated shape.

10. A lighting device of claim 1, further comprising an upholder, wherein:
one end of the upholder connects to the light head, and
the upholder supports a weight of the light head.

11. A lighting device of claim 10, wherein:
the upholder has an adjustable mechanical structure that adjusts a position of the light head, and
in the germicidal lighting mode, the first light head is positioned within 30 cm of a surface illuminated by the lighting device to enable more effective germicidal cleaning that otherwise.
